(19) United States
(12) Patent Application Publication
(10) Pub. No.: US 2005/0234540 A1
(43) Pub. Date: Oct. 20, 2005

(54) Dilatation systems and methods for left atrial appendage

(75) Inventors: Todd A. Peavey, Cambridge, MA (US); Andrzej J. Chanduszko, Chandler, AZ (US)

Correspondence Address: WILMER CUTLER PICKERING HALE AND DORR LLP, 60 STATE STREET, BOSTON, MA, 02109, US

(73) Assignee: NMT Medical, Inc., Boston, MA (US)

(21) Appl. No.: 11/077,176

(22) Filed: Mar. 10, 2005

Related U.S. Application Data
(60) Provisional application No. 60/552,821, filed on Mar. 12, 2004.

(51) Int. Cl. A61F 2/06

(52) U.S. Cl. 623/1.18; 623/1.42

(57) ABSTRACT

Devices and methods are provided for opening the entrance (ostium) of the left atrial appendage to increase blood flow and thereby reduce the likelihood of thrombus formation therein by decreasing blood stagnation. The device can include a stent, an expandable foam, or a balloon anchor component, and can be provided in such a way so as to leave no implant behind.

### CROSS REFERENCE TO RELATED APPLICATIONS

This application claims benefit to U.S. Provisional Patent Application Ser. No. 60/552,821 filed Mar. 12, 2004.

### FIELD OF THE INVENTION

The present invention relates generally to preventing the formation of thrombi in an anatomical appendage, such as the left atrial appendage.

### BACKGROUND

Arrhythmias are abnormal heart rhythms, which can cause the heart to function less effectively. Atrial fibrillation (AF) is the most common abnormal heart rhythm. In AF, the two upper chambers of the heart (i.e., the atria) quiver rather than beat and, consequently, fail to entirely empty of blood. If blood stagnates on the walls of the atria, it can form thrombi (i.e., clots). Under certain circumstances, these thrombi can re-enter circulation and travel to the brain, causing a stroke or a transient ischemic attack (TIA).

Research has indicated that as many as 90% of all thrombi formed during AF originate in a region known as the left atrial appendage (LAA). The LAA is a remnant of an original embryonic left atrium that develops during the third week of gestation. Referring to FIG. 1, the LAA 10 is located high on the free wall of a left atrium 12. Long and tubular in structure, the LAA 10 is connected to the left atrium 12 at a narrow junction 14, referred to as the ostium.

The precise physiological function of the LAA remains uncertain. Recent reports suggest it may maintain and regulate pressure and volume in the left atrium; modulate the hemodynamic response during states of cardiac stress; mediate thirst in hypovolemia; and/or serve as the site of release of the peptide hormone atrial natriuretic factor (ANF), which stimulates excretion of sodium and water by the kidneys and regulates blood pressure, and also the site of release of stretch sensitive receptors, which regulate heart rate, diuresis, and natriuresis.

It is believed that the physical characteristics of the LAA can cause the high rate of thrombus formation. Blood easily stagnates, and thereafter clots, in the long, tubular body of the LAA or at its narrow ostium. In contrast, the right atrial appendage (RAA), which is a wide, triangular appendage connected to the right atrium by a broad ostium, is infrequently the site of thrombus formation. Thrombus formation in the LAA is further promoted by the numerous tissue folds, referred to as crenellations 16, on its interior surface. Crenellations 16 are particularly hospitable to blood stagnation and clotting, especially when the heart is not functioning at maximum capacity. Thrombi formed in the LAA can re-enter the circulation upon conversion of AF to normal rhythm (i.e., cardioversion).

Currently, therapeutic protocols attempt to minimize the likelihood of thrombus formation associated with AF. Blood thinners, such as Warfarin (Coumadin), are frequently administered to AF patients. This administration is complicated by several factors. Warfarin is contraindicated for patients suffering from potential bleeding problems or ulcers. Also, Warfarin administration ideally begins approximately four weeks prior to cardioversion and continues for four weeks after cardioversion. This long course of treatment is often compromised due to emergency presentation and/or patient noncompliance.

Certain patient subsets are considered to be at an abnormally high risk of thrombus formation. Such patients include those over seventy-five (75) years of age, as well as those presenting with a history of thromboembolism, significant heart diseases, decreased LAA flow velocity, increased LAA size, spontaneous echogenic contrast, abnormal coagulation, diabetes mellitus, and/or systemic hypertension. For these high-risk patients, prophylactic intervention may be recommended. Current prophylaxes generally fall into three categories: (1) surgical ligation of the LAA (e.g., U.S. Pat. No. 6,561,969; U.S. Pat. No. 6,488,689); (2) implantation of an LAA occluder sufficient to prevent, or at least minimize, blood flow into the LAA (e.g., U.S. Pat. No. 6,551,303; U.S. Pat. No. 6,152,144; U.S. Patent Appln. No. 2003/0120337; U.S. Patent Appln. No. 2002/0111647; PCT/US02/23176) and (3) placement of a filter in the LAA ostium to prevent clots formed therein from re-entering the circulatory system (e.g., PCT/US03/02395; PCT/US02/17704).

Because it is not known exactly what physiological role the LAA plays, obliteration and occlusion are controversial. Reports suggest that obliterating the LAA may decrease atrial compliance and diminish ANF secretion.

While properly positioned filter devices prevent migration of thrombi into the circulatory system, they cannot inhibit thrombus formation within the LAA. Consequently, if the filter device is dislodged or ineffectively sealed against the LAA ostium (problems plaguing many current filter designs), clots held at the LAA ostium by the filter could be released into the circulation.

### SUMMARY

The present invention includes devices and methods for opening the entrance (ostium) of the LAA to increase blood flow and thereby reduce the likelihood of thrombus formation therein by decreasing blood stagnation. According to various embodiments, the device can include a stent, an expandable foam, or a balloon anchor component. The approaches described here are generally contrary to the known approaches described above, such as the occlusion, obliteration, or clamping approaches that seek to block or remove the LAA.

A stent in the LAA for helping to expand the opening can take a variety of forms. For example, it can have an open mesh wall as shown, which can be short with a small number of rows of cells, such as two or three or some other number of about five or less, or it may be longer. As indicated by FIG. 2 and by the use of the stent in this application, the stent would have a diameter or cross-section in an expanded form that would be greater than an unaltered internal dimension of the LAA. The stent can be made of nitinol, stainless steel, a nickel-cobalt based alloy (such as MP35N), or some other shape memory material, or it could be made of a polymer. It can include hooks for gripping at its desired location, it can include a mesh to perform some filtering function, and it can have different portions formed in a different way, such as with cells of different size, wires of different thicknesses, and wires with different treatments to have different stiffness and recovery force.

In another embodiment, the invention can include a method and apparatus for expanding the LAA through the use of an expandable material, such as a balloon or a foam that is used to open up the LAA. In the case of the foam, the foam can be biodegradable and dissolve after a period of time.

The invention thus includes a number of different embodiments of systems and methods which generally have the goal of reducing the formation of thrombi in the LAA, and more specifically, in most of the embodiments, the idea is to increase the flow of blood through the LAA to reduce the likelihood of thrombi being formed. In some cases, a frame (like a stent) is used to expand the ostium of the LAA and preferably the interior portion without providing any further blockage or filtering mesh, although filtering can be added. In the case of at least one embodiment, the LAA is expanded without any permanently implanted material.

Other features and advantages will become apparent from the following detailed description, drawings, and claims.

### BRIEF DESCRIPTION OF THE FIGURES

FIG. 1 is a schematic representation of the left atrium of a human heart, including an LAA.

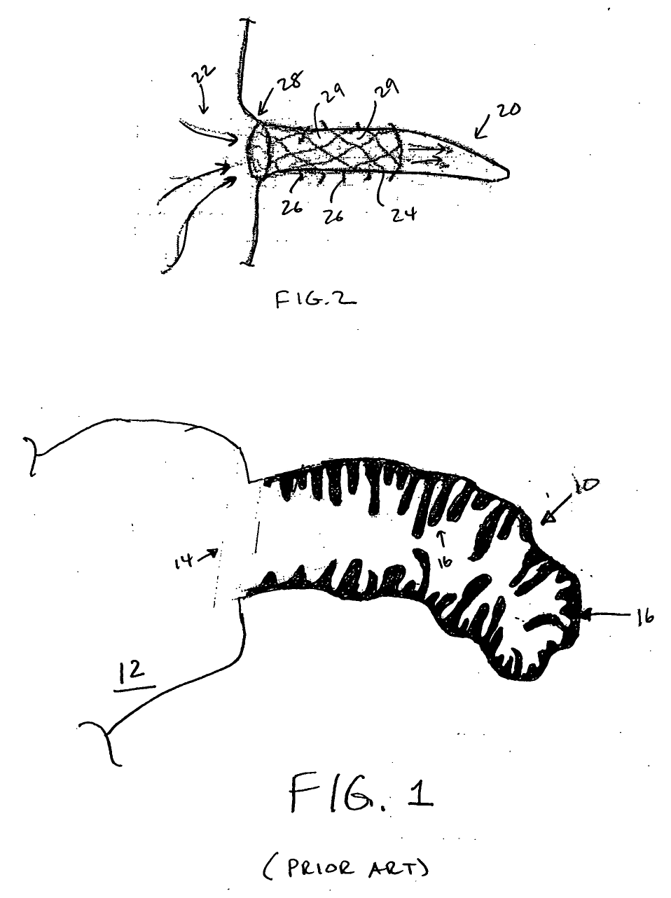

FIGS. 2-5 are perspective views of embodiments of the present invention using a form of a stent.

FIGS. 6A-6D are schematic representations of a method of expanding an LAA according to another embodiment of the present invention.

### DETAILED DESCRIPTION OF THE INVENTION

Embodiments of the present invention include devices and methods for modifying the LAA of a mammalian heart, including the human heart. These embodiments desirably reduce the likelihood of thrombus formation in the LAA during AF and, subsequently, stroke. In some embodiments, the devices expand the opening to the LAA, thereby increasing blood flow and minimizing blood stasis during AF. These modifications permit blood to enter and exit the LAA more easily during AF. Advantageously, because the device modifies, rather than eliminates, the LAA and potentially maintains LAA function, it overcomes potential drawbacks associated with obliterating or blocking the LAA.

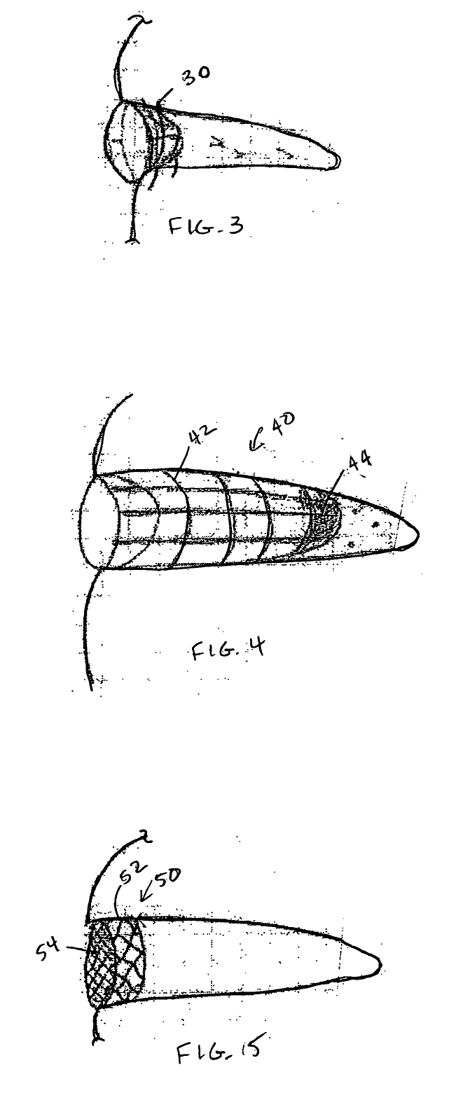

Referring to FIG. 2, a left atrial appendage (LAA) receives blood flow along arrows 22. LAA 20 has an ostium 28 into which is provided a frame, referred to here as stent 24. Stent 24 has a wire mesh with cells 29, as is generally known in the field of stents, which are generally known for their use in holding open arteries. In order to enlarge the LAA, a dimension of the stent in expanded form, such as a diameter or other cross-sectional dimension, would be larger than a corresponding dimension of the unaltered LAA. Stent 24 can have hooks 26 to help keep stent 24 in place within LAA 20. Using stent 24 at the ostium helps to expand the opening to the LAA, thereby increasing circulation into the LAA and reducing stagnation. This approach is different from occlusion or surgical closure approaches which are designed to reduce blood flow. By contrast, stent 24 is provided to open ostium 28 and/or interior portions of LAA 20 to increase blood flow, and can open them without any mesh or filter across the ostium.

The frame can be made of one of a variety of materials known for use in stents for other applications, such as a stainless steel, nitinol, a nickel-cobalt based alloy (such as MP35N), or other shape memory material, or the frame can be made of a polymer, including bioresorbable and shape memory polymers. In the case of a polymer or other material not easily visible with scanning equipment (such as X-ray), a radiopaque material, such as barium sulfate or tungsten, can be provided in or on the device. The frame is collapsible for delivery within a catheter, and then can expand on delivery in a manner that is generally known in the field of stents. As shown in FIG. 2, the frame can be in the opening and extend into the interior of the LAA, and can exert an outward pressure to help hold open the LAA without blocking the ostium and without a filtering mesh.

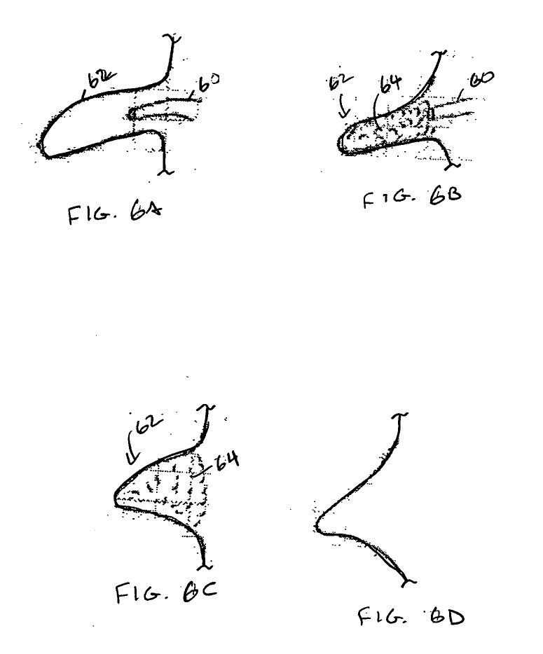

As is known in the field, the stent in its collapsed form may be delivered percutaneously via the vascular system by means of a catheter, such as, for example, catheter 60 shown in FIG. 6A. Upon reaching its intended location in the LAA and exiting the catheter, the stent expands, for example, as a result of release from an elastically compressed state within the catheter. Alternatively, if the stent is made of a shape memory material, the stent may expand when it assumes the body temperature of the subject. In another approach, an additional device, such as an inflatable balloon, is supplied to assist in enlarging the stent. The stent may also be inserted by direct surgical manipulation.

Referring to FIG. 3, in another embodiment, a stent 30 is referred to here as a short stent because it only has about 2 or 3 rows of openings or cells in the stent mesh. In this case, the device can still perform its function because it is propping open the ostium, and thereby increasing blood flow and reducing a static flow situation, also without any blockage or filtering in the ostium.

Referring to FIG. 4, a stent 40 has struts 42 that define a mesh in the frame. At a distal end of stent 40 is a region 44 with a finer mesh extending across the LAA and sized to help block clots that may form in the LAA to prevent them from being released from the LAA back into circulation. Referring to FIG. 5, a shallow stent 50 has a set of wires 52 that define a mesh that can be a small number of rows of openings, such as 2 to 4 rows. At the opening of the LAA, a fine mesh 54 extends across the ostium to serve as a filter for clots that may be formed inside the LAA. In these cases, the ostium is opened, but a mesh is used for filtering, although the embodiment of FIG. 4 also has nothing else in the ostium, and the embodiments of FIGS. 2 and 3 show a stent with no filter and no blocking piece across the ostium.

Referring to FIG. 6A-6D, a method for expanding the ostium is described. FIG. 6A shows the introduction of a catheter 60 into a LAA 62. The catheter is used to introduce a foam or other expandable material 64 into LAA 62 as shown in FIG. 6B. FIG. 6C illustrates LAA 62 with the foam 64 in an expanded state after catheter 60 has been removed. Foam 64 is preferably made of a biodegradable or bioresorable polymer that completely dissolves over time. As shown in FIG. 6D, after foam 64 has been resorbed or dissolved, the configuration of the LAA has changed into one that is more triangular than the elongated LAA of FIG. 6A, with no permanent material or implant remaining. Suitable foam materials include polyvinyl alcohol, silicone, or polyurethane. Alternatively, a balloon could be used to expand the opening and then be removed.

The devices described herein may be used with anti-thrombogenic compounds, including but not limited to heparin (ionic or covalently-bound) and peptides, to reduce thrombogenicity of the device and/or to enhance the cellular ingrowth of the cardiac tissue following deployment of the device in vivo. Similarly, the devices described herein may be used to deliver other drugs or pharmaceutical agents (e.g., growth factors or antibodies). The anti-thrombogenic compounds and/or pharmaceutical agents may be included in the device in several ways, including impregnation or coating of the stent component or included in a foam. Further, the devices described herein may include radiopaque fillers for x-ray visualization, cells to promote biocompatibility, echogenic coatings, lubricious coatings, and/or hydrogels.

Having described preferred embodiments of the invention, it should be apparent that various modifications may be made without departing from the spirit and scope of the invention. Any of the stent-like embodiments can be further coated with an antiplatelet or anticoagulant to produce a drug eluting stent. If a stent is made from a shape memory material like nitinol, portions of the stent can be treated differently to produce different transition temperature, or portions can have different cell sizes and/or different material thicknesses at different locations. These variations can cause the amount of expansion to vary, and/or otherwise alter the stiffness or recovery force in desired locations. For example, it may be desirable to have the portion of the stent near the ostium have more expansion force.

Having described certain embodiments, it should be apparent that modifications can be made without departing from the scope of the invention as defined by the appended claims. For example, certain materials have been stated. Although other suitable materials could be used.

1. A method of enlarging a portion of a left atrial appendage (LAA) of a heart of a subject, the method comprising:
inserting an expandable stent into the LAA, wherein the stent in expanded form has a dimension greater than an unaltered internal dimension of the LAA, the stent enlarging the internal dimension of the LAA.

2. The method of claim 1, wherein the stent comprises a wall including an open mesh.

3. The method of claim 1, wherein the stent comprises a shape memory material.

4. The method of claim 1, wherein the stent further comprises a mesh extending across a proximal end of the inserted stent, the mesh sized to block the passage of thrombi.

5. The method of claim 1, wherein the stent further comprises a mesh extending across a distal end of the inserted stent, the mesh sized to block the passage of thrombi.

6. The method of claim 1, wherein the stent includes at least one of a radio-opaque material, cells to promote biocompatibility, echogenic coatings, lubricious coatings, and hydrogels.

7. The method of claim 1, wherein the stent includes at least one of an antiplatelet agent and an anticoagulant.

8. The method of claim 1, wherein the stent is treated to exert an expansion force on an interior wall of the LAA that varies along a length of the stent.

9. The method of claim 1, further comprising providing a catheter for percutaneous transluminal positioning of the stent.

10. The method of claim 1, wherein enlarging an internal dimension of the LAA reduces the risk of formation of thrombi within the LAA.

11. The method of claim 1, wherein the internal dimension of the LAA is a diameter of the ostium of the LAA.

12. The method of claim 1, wherein the stent comprises two to four rows of cells in an open mesh.

13. The method of claim 1, wherein the stent comprises more than four rows of cells in an open mesh.

14. A method of enlarging a diameter of an ostium of a left atrial appendage (LAA) of a heart of a subject, the method comprising:
inserting a foreign object into the LAA to enlarge the diameter of the ostium of the LAA without blocking the ostium.

15. The method of claim 14, wherein the ostium is expanded without leaving an implant within the LAA.

16. The method of claim 15, wherein the foreign object is an inflatable balloon that can be expanded and then contracted and withdrawn.

17. The method of claim 15, wherein the foreign object is a foam material.

18. The method of claim 17, wherein the foam material is removed by biodegradation.

19. The method of claim 17, wherein the foam material comprises a material selected from the set consisting of polyvinyl alcohol, silicone, and polyurethane.

20. The method of claim 14, wherein the foreign object further comprises at least one of an antiplatelet agent and an anticoagulant.

21. The method of claim 14, wherein the foreign object includes a stent with a wire mesh.

22. An apparatus for enlarging a left atrial appendage (LAA) of a heart, the apparatus comprising:
a stent having a diameter larger than a diameter of an ostium of the LAA, for use in expanding the ostium of the LAA.

23. The apparatus of claim 22, wherein the stent comprises a shape memory material.

24. The apparatus of claim 22, wherein the stent further comprises a mesh extending across a proximal end of the inserted stent, the mesh sized to block the passage of thrombi.

25. The apparatus of claim 22, wherein the stent further comprises a mesh extending across a distal end of the inserted stent, the mesh sized to block the passage of thrombi.

26. The apparatus of claim 22, wherein the stent includes at least one of a radio-opaque material, cells to promote biocompatibility, echogenic coatings, lubricious coatings, and hydrogels.

27. The apparatus of claim 22, wherein the stent comprises two to four rows of cells in an open mesh.

28. The apparatus of claim 22, wherein the stent comprises more than four rows of cells in an open mesh.
